(19) United States
(12) Patent Application Publication
(10) Pub. No.: US 2001/0047398 A1
(43) Pub. Date: Nov. 29, 2001

(54) Managing chemical information and commerce

(76) Inventor: Stewart D. Rubenstein, Newton Center, MA (US)

Correspondence Address: HALE AND DORR, LLP, 60 STATE STREET, BOSTON, MA, 02109

(21) Appl. No.: 09/796,007

(22) Filed: Feb. 28, 2001

Related U.S. Application Data
(60) Provisional application No. 60/186,156, filed on Feb. 29, 2000.

(51) Int. Cl. G06F 15/16

(52) U.S. Cl. 709/218; 709/206

(57) ABSTRACT

A description of a chemical substance is acquired and a user's selection of a user interface menu item is detected. The user interface menu item pertains to a Web server having access to data for chemical substances. Based on the detection, a request is submitted to the Web server for information based on the description of the chemical substance. A response is received from the Web server that includes data pertaining to the chemical substance. Output data is derived from the data pertaining to the chemical substance.

This application claims the benefit of United States Provisional Application Serial No. 60/186,156 entitled “MANAGING CHEMICAL PRODUCT COMMERCE”, filed on Feb. 29, 2000, which is incorporated herein by reference.

### BACKGROUND OF THE INVENTION

This application relates to managing chemical information and commerce.

A chemistry oriented application program such as a chemical drawing program allows chemical information to be handled as computer data by a computer. A chemical drawing program typically allows a user to cause chemical structural information and chemical reaction information to be displayed on a computer screen and printed out on a computer printer. If a user has chemical information in the application program that is relevant to another application program, the user typically can use the cut and paste capabilities of the application program to copy the relevant information to the other application program. If one of the application programs is a Web browser interacting with a Web server, the user typically can use the cut and paste capabilities to share information between the Web browser and the other application program, and can thereby, in a limited way, share information between the Web server and the other application program. However, existing chemically oriented application programs provide inadequate abilities to access chemical information that is stored in different formats or in remote locations.

### SUMMARY OF THE INVENTION

Methods and systems are provided for managing chemical information and commerce. In particular, an external query capability allows a chemistry oriented application program to query an external entity such as a chemical information external database using the Hypertext Transfer Protocol (“HTTP”), to produce information management and commerce management results not otherwise realizable by the application program.

Different aspects of the invention allow one or more of the following. An application program having chemical information can submit that information in a query to a remote chemical database and receive and incorporate a response from the database, without requiring the user to run or exercise another application program such as a Web browser. A user can provide chemical information in one form (e.g., structural) to an application program which can submit the information to another entity such as a remote database in a different form (e.g., textual) that is meaningful to the other entity, and that can lead to retrieval of information that is useful to the user. The capabilities of a chemistry oriented software tool can be linked to, and thereby applied to, a remote organized set of chemical information to produce analytical and commercial results such as reports and purchasing lists based on the remote organized set of chemical information.

Other features and advantages will become apparent from the following description, including the drawings, and from the claims.

### BRIEF DESCRIPTION OF THE DRAWINGS

FIG. 1 is a block diagram of a computer-based system.

FIGS. 2-11, 16 are illustrations of output displays that may be produced by the computer based system.

FIGS. 12A-15 are illustrations of data forms that may be used in the computer based system.

### DETAILED DESCRIPTION

A chemistry oriented application program has an external query capability that allows the program to query an external entity such as a chemical information external database.

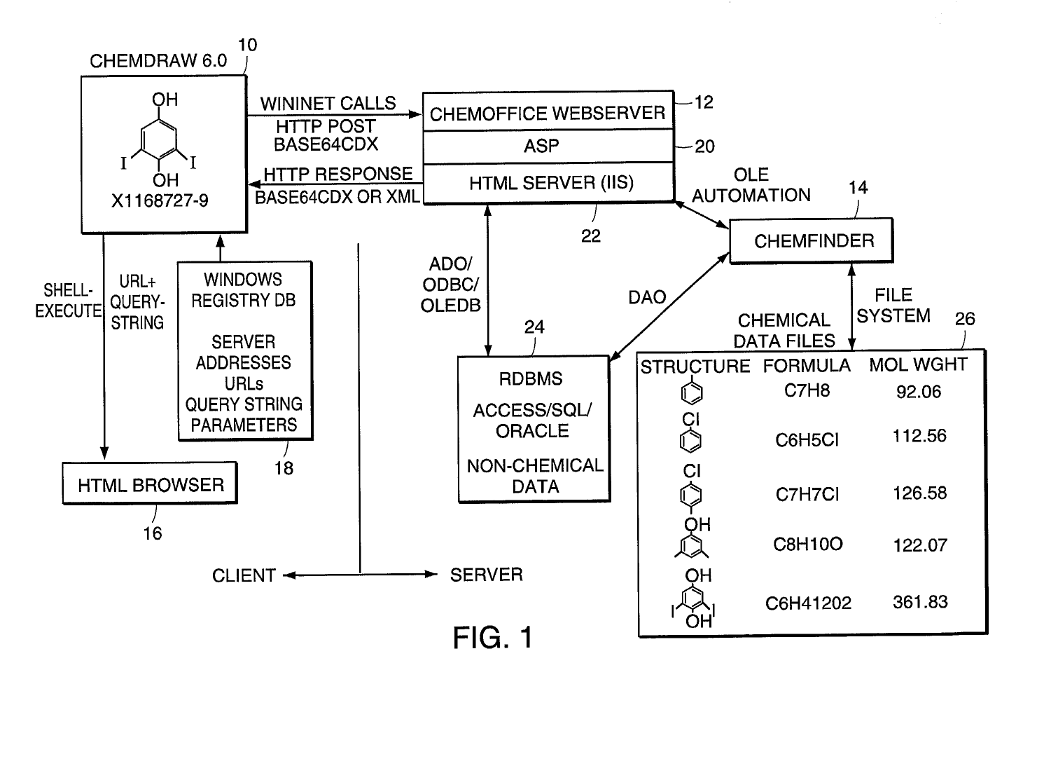

FIG. 1 illustrates an example system in which a chemical drawing application program 10 (in this example, ChemDraw 6.0, provided by CambridgeSoft Corporation of Cambridge, Mass.) serves as the application program and uses the external query capability to acquire information via HTTP from a Web server 12 (in this example, ChemOffice WebServer, provided by CambridgeSoft Corporation of Cambridge, Mass.) linked to a chemical information database 14 (in this example, ChemFinder, provided by CambridgeSoft Corporation of Cambridge, Mass.). Although these particular chemistry oriented programs are provided to illustrate the present invention, other applications programs, Web servers, databases, and other programs may be used as well or instead.

In the example system, which is a Microsoft Windows implementation using ChemDraw, ChemDraw can, if necessary, invoke a Web browser 16 (e.g., see the third and fourth examples below) and can retrieve information including server addresses, uniform resource locators (URLs), and query string parameters from a Windows registry database (“Windows registry”) 18. ChemDraw can use WinIet calls to post HTTP requests to the ChemOffice Webserver, and receives responses in HTTP form. WinIet, which is supplied as part of the Windows operating system, is a set of functions (also known as calls, procedures, and subroutines) in an Internet application programming interface (API) that may be implemented as dynamic link library file (DLL). A DLL provides a library of functions that applications link to and call as regular function calls.

The ChemOffice WebServer uses Active Server Pages (“ASP”) software 20 and Hypertext Markup Language (HTML) server software known as Internet Information Server (“IIS”) 22 to interact with ChemFinder via Object Linking and Embedding (OLE) Automation, and with a relational database management system (“RDBMS”) 24. (Other interaction mechanisms may be used in place of or in addition to OLE Automation.) Web pages managed by ChemOffice WebServer may be formatted according to HTML or XML (Extensible Markup Language). Additionally, Web pages may be formatted as, for example, active server page text files (“ASP files”) compatible with the ASP software. An ASP file may rely on a combination of HTML or XML and a scripting language such as VBScript or JavaScript.

The interaction with the RDBMS is implemented using one or more data access technologies such as Open Database Connectivity (ODBC), Active Data Objects (ADO), and OLEDB. ODBC is a standard for accessing different database systems from a high level programming language application, and enables applications to submit statements to ODBC using structured query language (SQL), which statements are then translated to the particular SQL commands that are used by the specific underlying database product.

The RDBMS includes non-chemical data such as substance prices. ChemFinder is able to retrieve chemical information such as chemical structure data (“chemical structure”), chemical formulas, and molecular weights from chemical data files 26, and interacts with the RDBMS using, e.g., ADO technology. According to ADO, data from a database is mapped into active data objects which perform the actual queries to the database. This approach provides substantial abstraction by limiting exposure only to the resultant objects.

ChemDraw communicates with an external chemical database server as now described with reference to multiple examples including an example illustrating how ChemDraw may be used to purchase chemicals online at the Available Chemicals Xchange web site (ChemACX.com). ChemDraw can initiate a database query based on either a chemical structure drawing or a simple text string. (Other application programs may use other data formats.) The response provided by the chemical database server can be a simple text response, a chemical structure, or a complex record set containing a combination of chemical and non-chemical data. The process by which ChemDraw handles a query differs slightly depending on the request/response method that is available.

In a first example, a chemical structure drawing is used to retrieve a plain text response. Using ChemDraw, the user creates a drawing of a molecule which is stored as chemical structure by ChemDraw in a native binary format known as cdx.

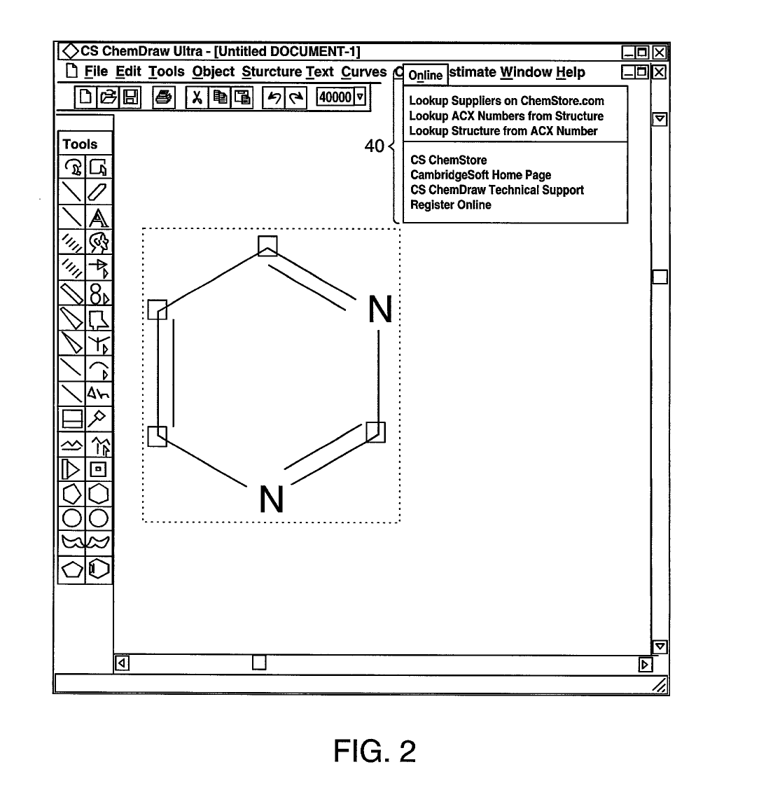

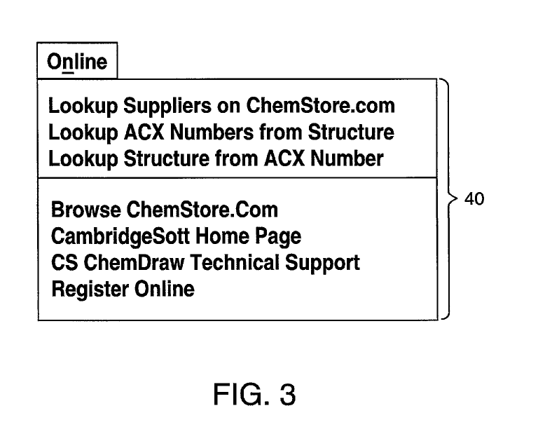

When the user selects “Look up ACX number from structure” from an online menu 40 (FIGS. 2-3), which is a pulldown menu in ChemDraw, ChemDraw converts the chemical structure data from the cdx format to an ASCII text representation. The cdx-to-ASCII conversion is accomplished via a base64 encoding process with a subsequent URL encoding for safe transport over the HTTP protocol. Base64 is an encoding scheme defined by the Multipurpose Internet Mail Extensions (MIME) standard, and is defined to provide robustness to binary data that is expected to confront transformations while traversing the Internet. Base64 encoding converts binary data into alphanumeric characters. For example, binary data such as “110110001011010100000101” may be encoded as textual data “sLUF” which may be transmitted across the Internet.

ChemDraw uses configuration information stored in the Windows registry database to derive a target URL, i.e., a URL pointing to a remote chemical database server. Using the URL derived from the registry information and the URL encoded ASCII text representation of the chemical structure drawn by the user, ChemDraw uses WinIet to post an HTTP request. The converted chemical structure data is passed in the body of the HTTP request.

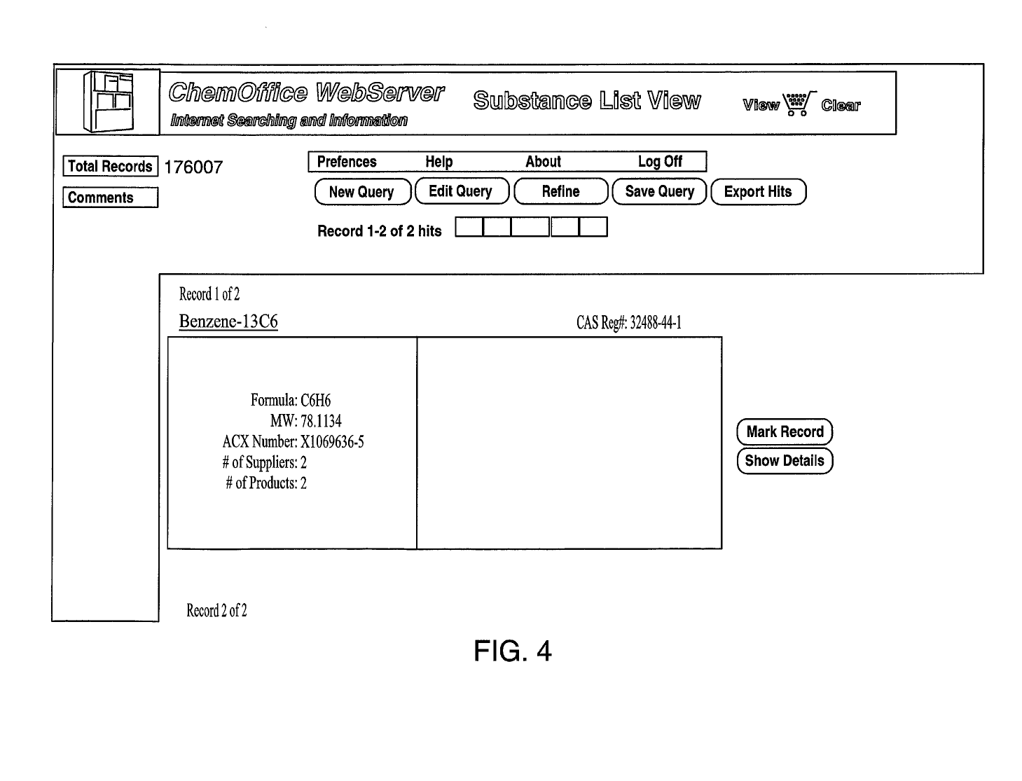

The remote chemical database server responds with a comma delimited text string containing one or more ACX numbers (e.g., ACX number X1069636-5 shown in FIG. 4) corresponding to the chemical structure drawn by the user. (ACX numbers are product identifiers described in two commonly assigned co-pending applications filed May 5, 2000: 09/565,085 entitled “DERIVING PRODUCT INFORMATION”, and 09/565,810 entitled “MANAGING PRODUCT INFORMATION”, which applications are incorporated herein by reference.)

ChemDraw captures the server's response by reading the HTTP response.

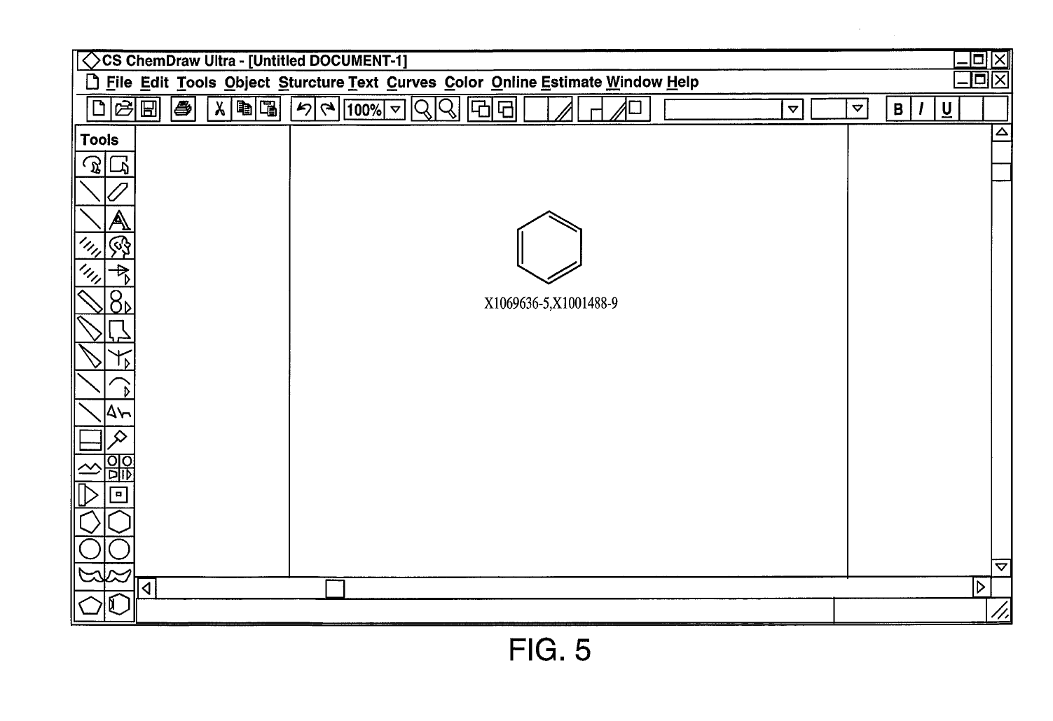

As shown by example in FIG. 5, the ACX numbers received from the server are displayed by ChemDraw on the ChemDraw canvas in a text box displayed near, e.g., immediately below, the user's chemical structure drawing.

In a second example, a plain text input value is used to retrieve a chemical structure drawing. In particular, a reverse version of the first example above can be accomplished by providing a valid ACX number and retrieving the corresponding chemical structure. In this example, the user selects “Lookup structure from the ACX number” in the ChemDraw 6.0 online menu.

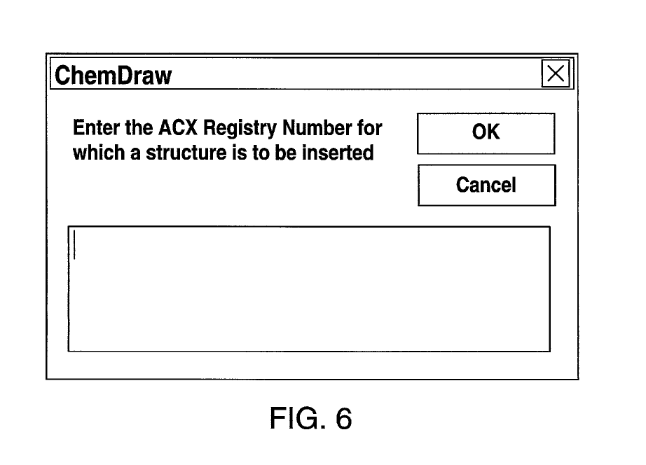

A dialog box shown by example in FIG. 6 opens and prompts the user to enter an ACX number.

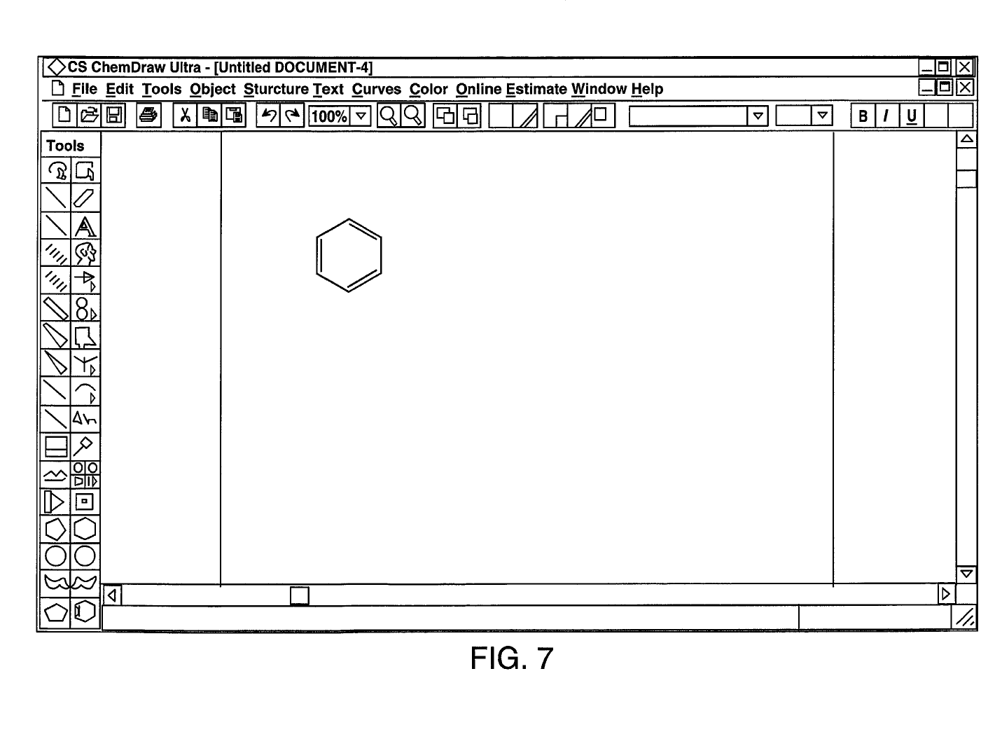

ChemDraw retrieves target URL information from the Windows registry and builds an HTTP request. In this case, the payload of the HTTP request is a string containing the ACX number, which is appended to the URL as a query string. The HTTP response returned by the remote server contains a URL encoded base64 text representation of a chemical structure. ChemDraw decodes the text representation and displays the structure on its canvas as a native ChemDraw picture shown by example in FIG. 7.

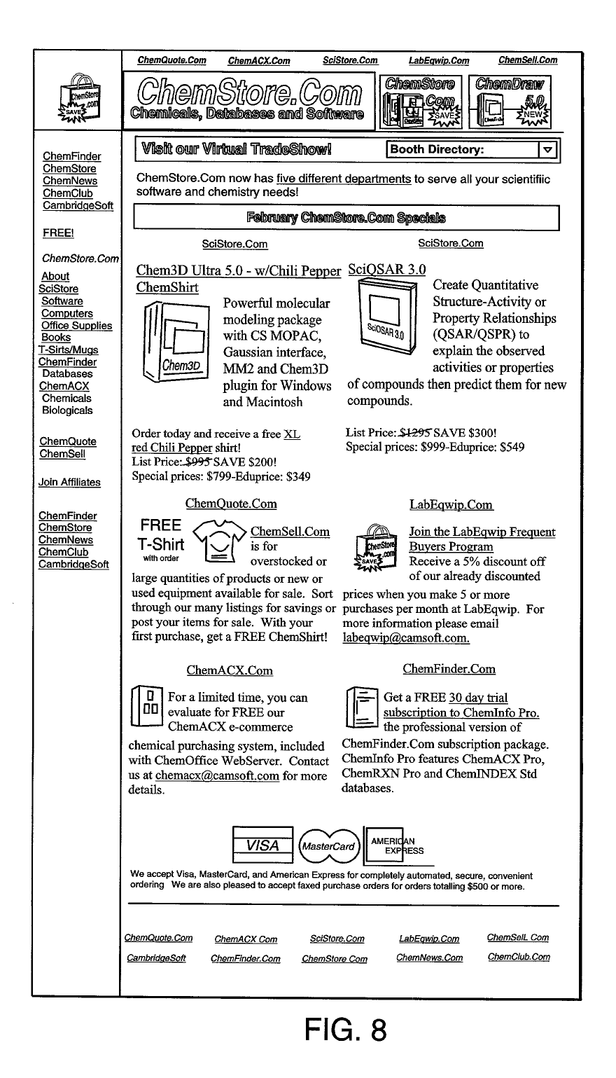

In a third example, an external HTML browser session is initiated with a remote server. Although ChemDraw is able to access remote servers by directly managing HTTP calls, ChemDraw can also delegate interactions with the server to an external HTML browser. A user selects a “Browse ChemStore.Com” online menu item in ChemDraw, which causes the default HMTL browser to be launched on the user's computer and directed to load a Web page from ChemStore.com as shown by example in FIG. 8.

ChemDraw proceeds in the third example as follows. ChemDraw builds a URL from configurable data stored in the Windows registry. ChemDraw issues a call to ShellExecute with the target URL as a parameter, which causes the user's default browser to open and load the page specified by the URL. (In Microsoft Windows, the ShellExecute function either launches the specified application program, or, if the program is already executing, makes the program the current window.)

Complex URLs corresponding to user specific server pages can be built by incorporating data received from previous queries using techniques described in the first and second examples above. For example, a specific page from a chemical supplier's catalog can be retrieved based on a structure of a molecule drawn by the user.

In a fourth example, a user accomplishes online purchasing of chemicals by starting from ChemDraw. The first and second examples above illustrate how ChemDraw can send or receive a chemical structure or text data to or from a remote chemical database server and display the results directly in ChemDraw's drawing area. When the results of a query are expected to be lengthy or complex, ChemDraw causes a standard HTML browser to handle the display instead.

In this example, chemicals are purchased online from ChemACX.com. As described in the first example above, the user draws a molecule and selects the “Lookup suppliers from ChemACX.com” online menu item.

The chemical structure drawn by the user is used in this example to identify the substance for which purchasing information is to be fetched. Thus, the procedure is as described in the first example above, except that the value returned by the server is a substance identifier that is not meaningful to the end user, and therefore is not displayed, but is used as an intermediate value for querying a Web based catalog.

Specifically, the substance identifier is posted to a remote server to obtain the desired purchasing information. The requested result set is to be displayed in an external browser window. Accordingly, ChemDraw uses techniques described above in the third example to build a URL pointing the specific catalog page at ChemACX.Com which contains information about the desired substance. This URL is built from server target information stored in the Windows registry database and from the unique substance identifier fetched from the server in the initial step described above.

ChemDraw opens the user's default external browser and automatically causes the browser to load the appropriate catalog page. The user can use the browser to navigate the available purchasing information and initiate the online ordering process from within the browser.

In a specific implementation in which the user is presented with a list of suppliers and prices in a Web browser window, the user can then check off the supplier and desired quantity, which causes a corresponding shopping cart construct to be created, ready for submission for purchase completion.

The online menu may also be configured to retrieve other pieces of data based on a selected structure, such as specific compound identification numbers from specific manufacturers.

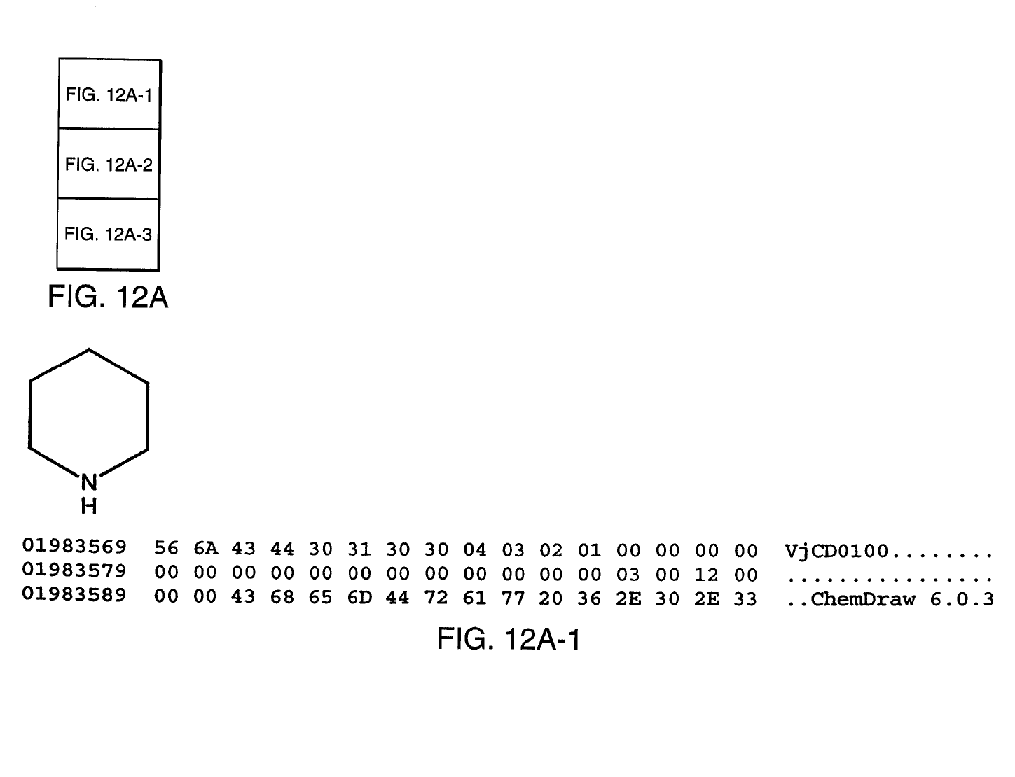

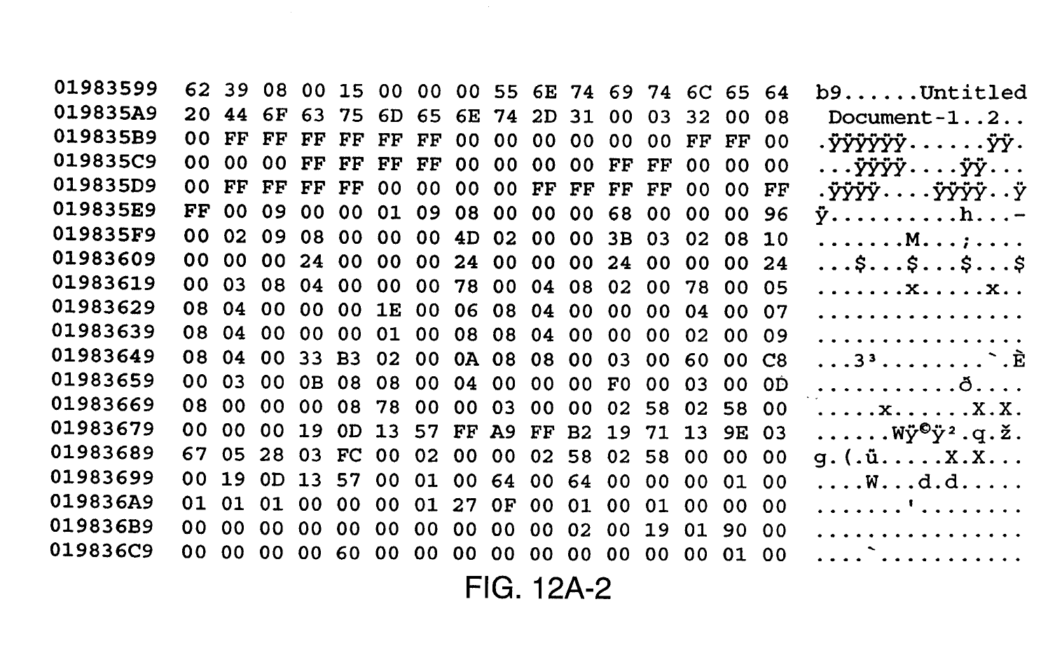

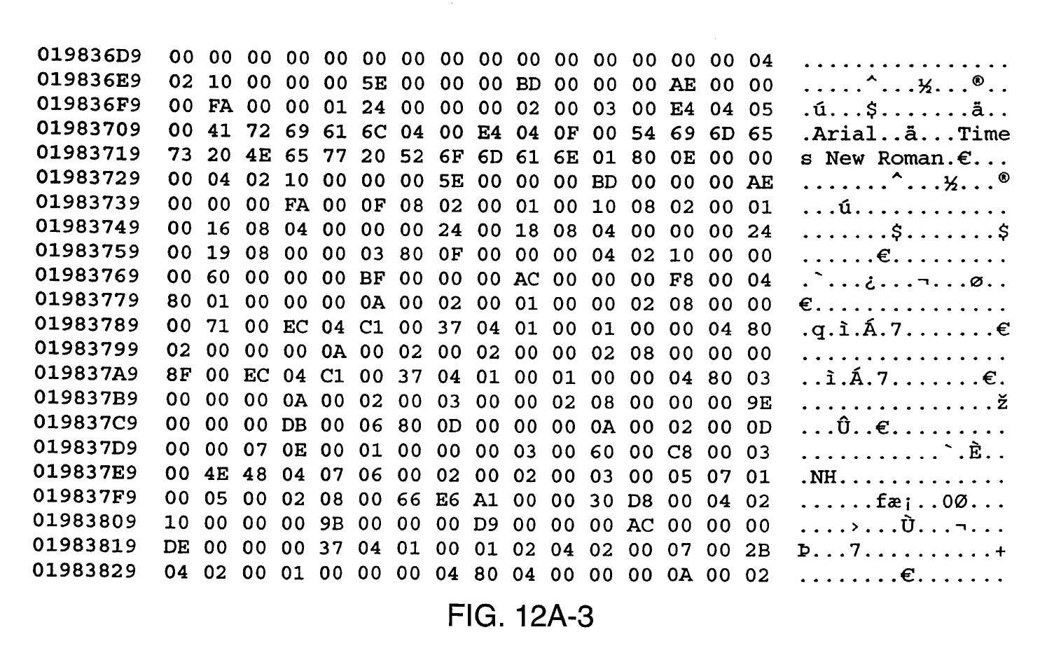

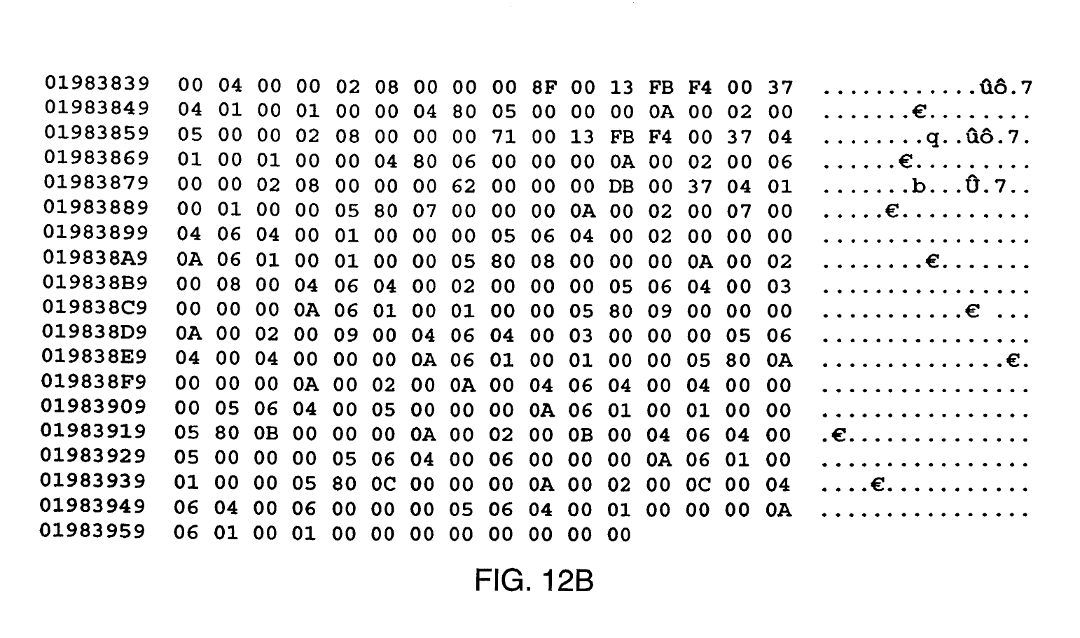

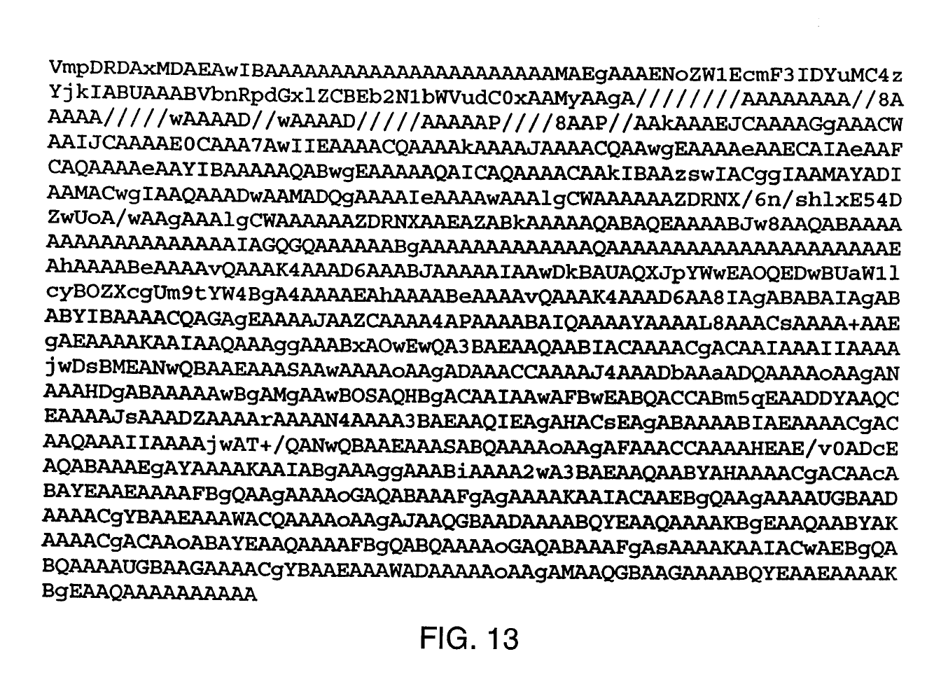

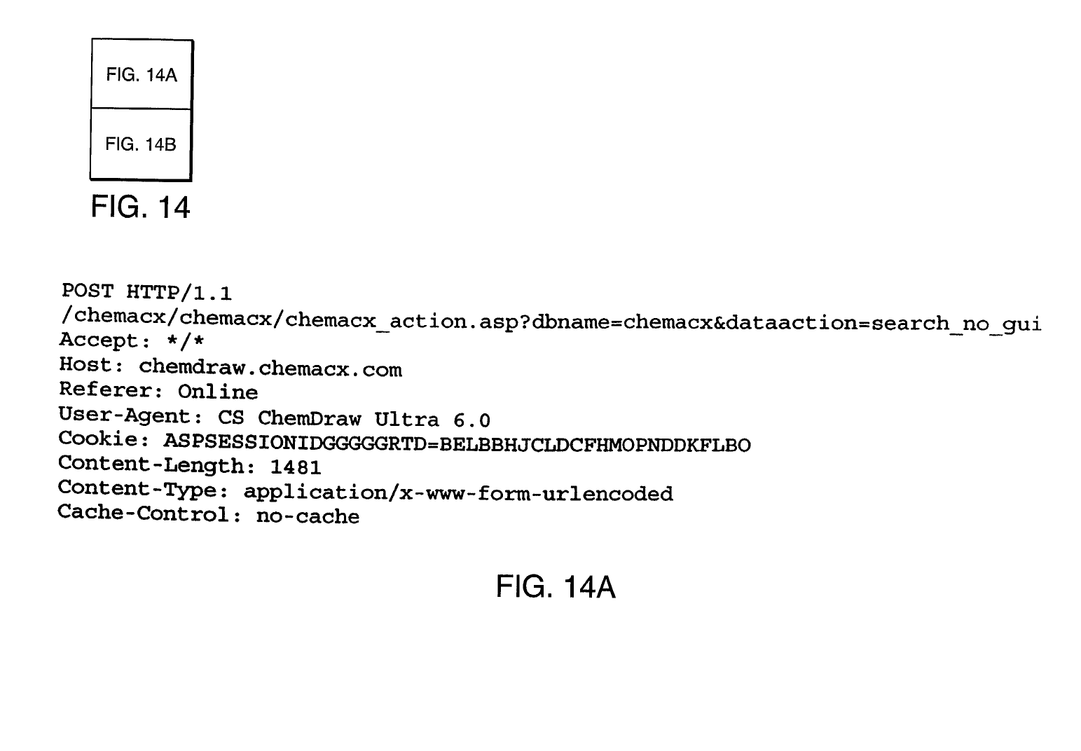

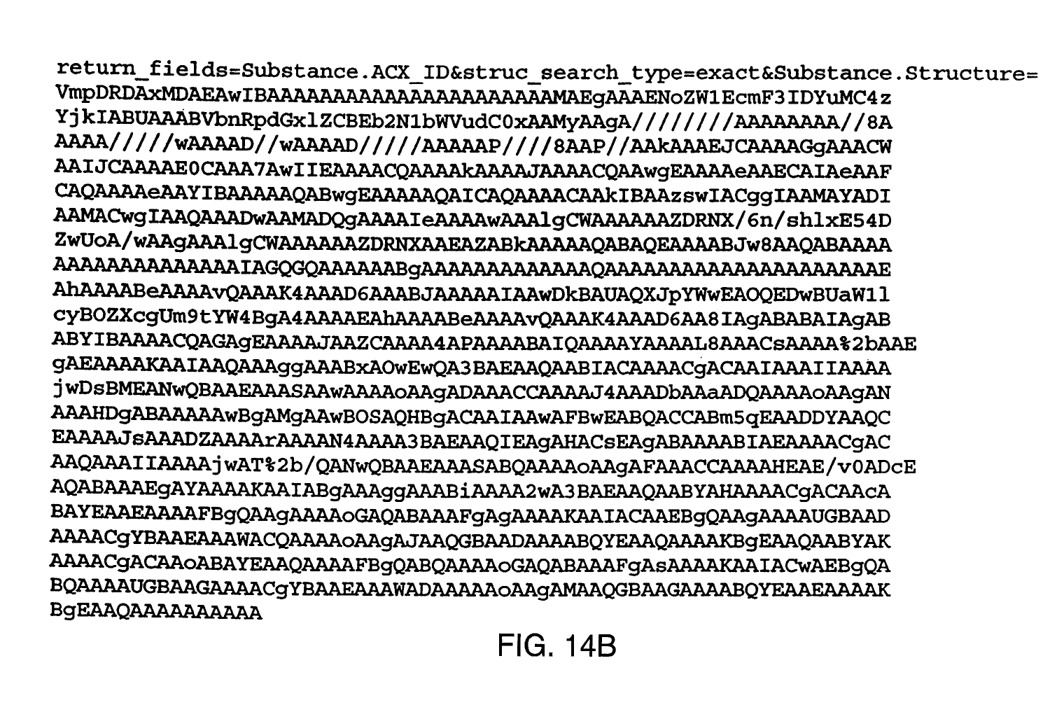

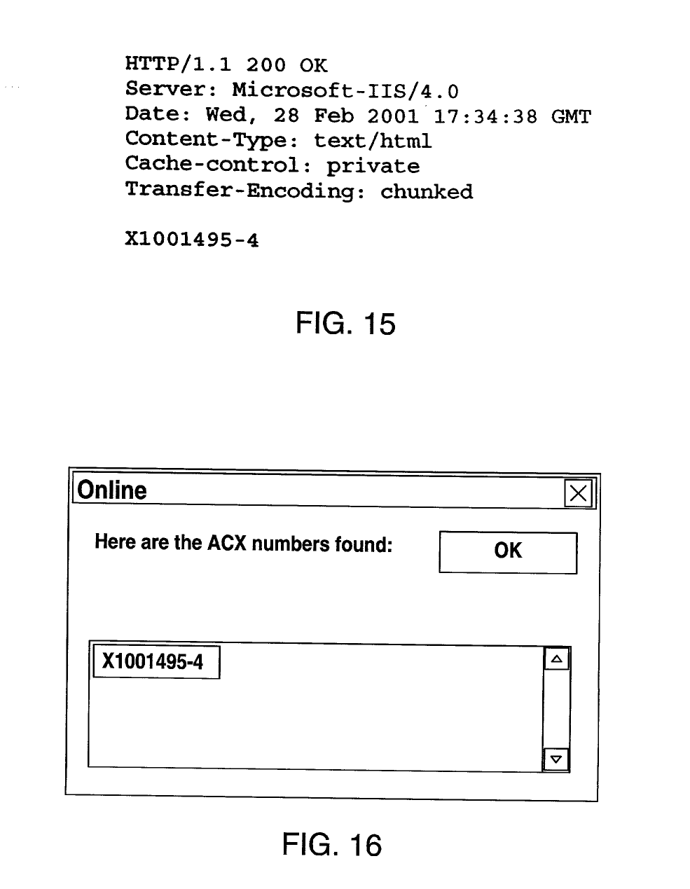

FIGS. 12A-16 illustrate a specific example of data transformations referenced above, with respect to an example interaction when a user draws a structure and selects the “Find ACX Numbers from Structure” online menu item. FIGS. 12A-12B illustrate a chemical structure and its corresponding binary CDX form. FIG. 13 illustrates the base64 encoded form corresponding to FIGS. 12A-12B. FIG. 14 illustrates the encoded request corresponding to FIG. 13. FIG. 15 illustrates the response from the server corresponding to FIG. 14. FIG. 16 illustrates a display provided to the user corresponding to FIG. 15.

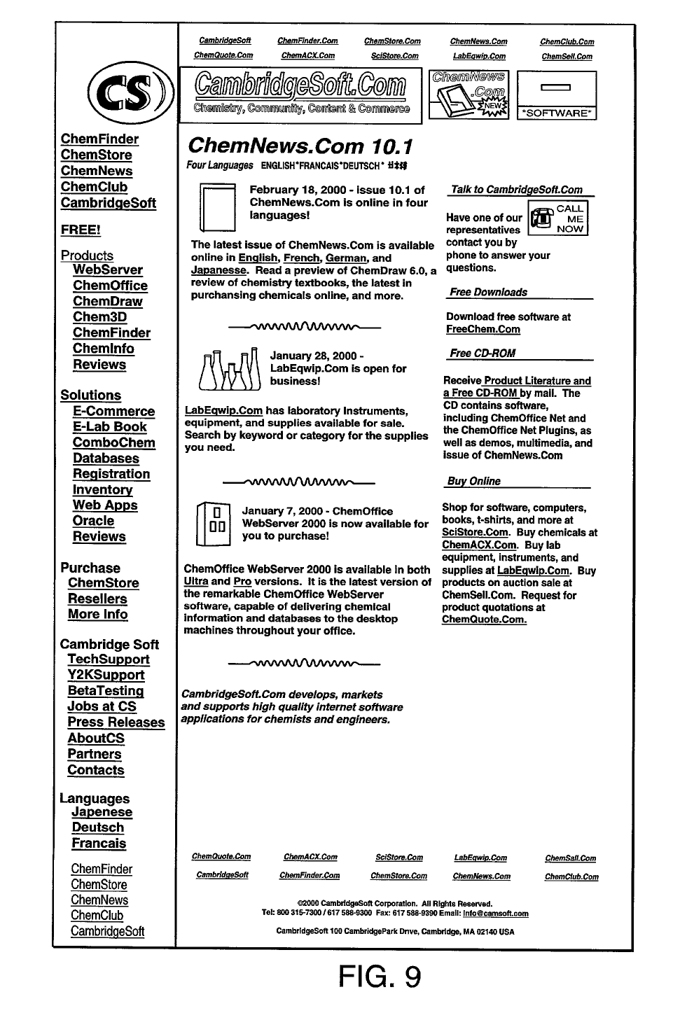

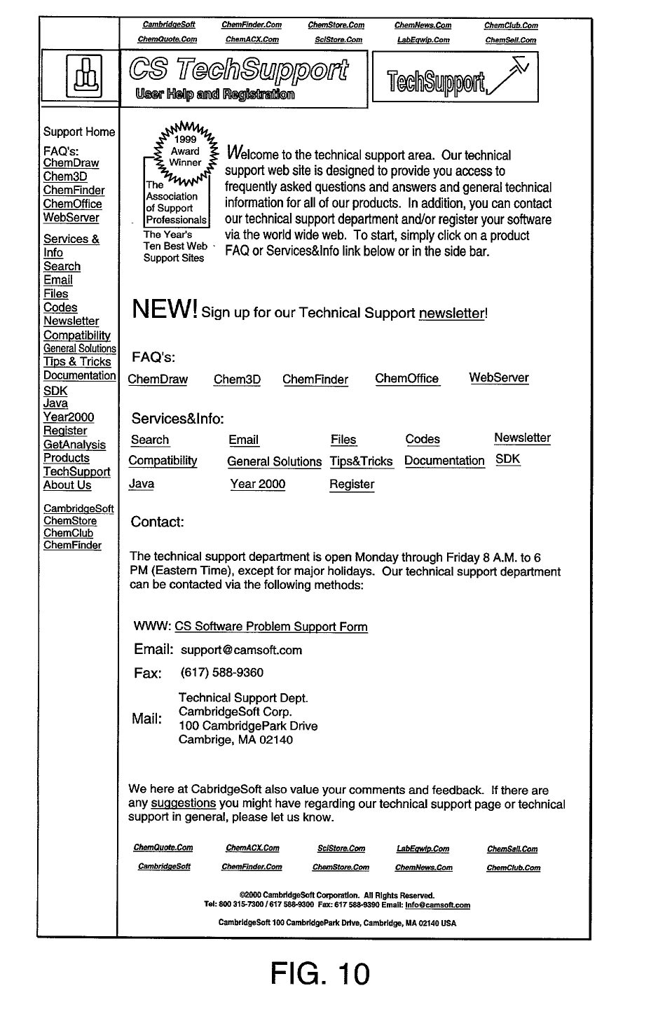

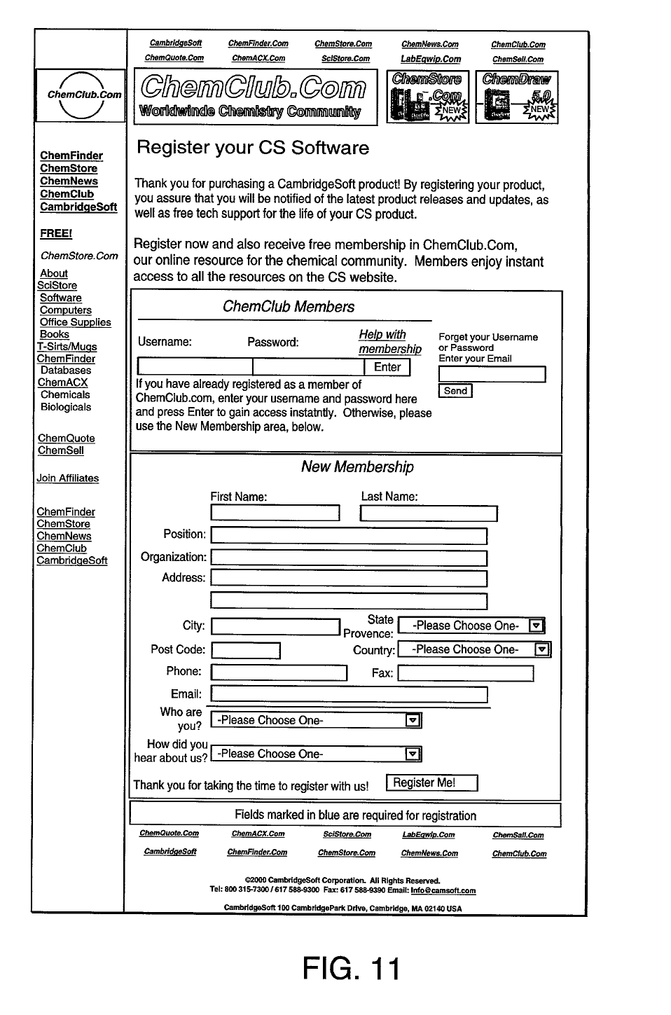

FIGS. 9-11 illustrate examples of other pages that may be retrieved by selection of, respectively, the “CambridgeSoft Home Page”, “CS ChemDraw Technical Support”, and “Register Online” online menu items shown in FIGS. 2-3.

All, or a portion, of the procedures described above may be implemented in hardware or software, or a combination of both. In at least some cases, it is advantageous if the technique is implemented in computer programs executing on one or more programmable computers, such as a personal computer running or able to run an operating system such as Unix, Linux, Microsoft Windows 95, 98, 2000, or NT, or Macintosh OS, that each include a processor, a storage medium readable by the processor (including volatile and non-volatile memory and/or storage elements), at least one input device such as a keyboard, and at least one output device. Program code is applied to data entered using the input device to perform the technique described above and to generate output information. The output information is applied to one or more output devices such as a display screen of the computer.

In at least some cases, it is advantageous if each program is implemented in a high level procedural or object-oriented programming language such as Perl, C, C++, or Java to communicate with a computer system. However, the programs can be implemented in assembly or machine language, if desired. In any case, the language may be a compiled or interpreted language.

In at least some cases, it is advantageous if each such computer program is stored on a storage medium or device, such as ROM or optical or magnetic disc, that is readable by a general or special purpose programmable computer for configuring and operating the computer when the storage medium or device is read by the computer to perform the procedures described in this document. The system may also be considered to be implemented as a computer-readable storage medium, configured with a computer program, where the storage medium so configured causes a computer to operate in a specific and predefined manner.

Other embodiments are within the scope of the following claims. For example, a more general range of services may be provided. A query may be performed on local or remote databases, and may be used in the derivation of a non-exact query or a non-structural query. For instance, in the case of ChemDraw which provides property prediction facilities, such facilities may be used to predict physical characteristics of a molecule of interest, and local or remote databases may be searched for substances having similar properties. The online menu may be used in another application program such as a spreadsheet program. For instance, in the case of the spreadsheet program, the program may store a shopping list of chemical products, and the online menu may include a selectable item for retrieving price information for each listed product from a remote chemical information database and embedding the retrieved price information in the shopping list.

What is claimed is:

1. A method for use in managing chemical information, comprising:
acquiring a description of a chemical substance;
detecting a user's selection of a user interface menu item pertaining to a Web server having access to data for chemical substances;
based on the detection, submitting, to the Web server, a request for information based on the description of the chemical substance;
receiving a response from the Web server, the response including data pertaining to the chemical substance; and
deriving output data from the data pertaining to the chemical substance.

2. The method of claim 1, wherein the description of the chemical substance includes a structural description of the chemical substance.

3. The method of claim 1, wherein the description of the chemical substance includes a textual description of the textual substance.

4. The method of claim 1, wherein the output data includes structural output data.

5. The method of claim 1, wherein the output data includes textual output data.

6. The method of claim 1, wherein the output data includes commercial product data.

7. The method of claim 1, wherein the user interface item is provided by a chemistry oriented application program.

8. The method of claim 7, wherein the chemistry oriented application program includes a chemical drawing program.

9. The method of claim 1, further comprising formatting at least a portion of the request for information according to the HTTP protocol.

10. The method of claim 1, further comprising decoding at least a portion of the response from the Web server according to the HTTP protocol.

11. The method of claim 1, further comprising presenting a user interface window that includes a display corresponding to the description of the chemical substance and another display derived from the output data.

12. The method of claim 1, further comprising causing a new Web browser window to be displayed; and
causing a display derived from the output data to be displayed in the new Web browser window.

13. Computer software, residing on a computer-readable storage medium, comprising a set of instructions for use in a computer system to help cause the computer system to manage chemical information, the instructions causing the system to:
acquire a description of a chemical substance;
detect a user's selection of a user interface menu item pertaining to a Web server having access to data for chemical substances;
based on the detection, submit, to the Web server, a request for information based on the description of the chemical substance;
receive a response from the Web server, the response including data pertaining to the chemical substance; and
derive output data from the data pertaining to the chemical substance.

14. A system for use in managing chemical information, comprising:
a Web server having access to data for chemical substances; and
a chemistry oriented application program providing a user interface menu item linked to the Web server.
